(19) United States
(12) Patent Application Publication
(10) Pub. No.: US 2016/0129279 A1
(43) Pub. Date: May 12, 2016

(54) WEARABLE THERAPEUTIC LIGHT SOURCE

(71) Applicant: Akari Systems, Inc., Sunnyvale, CA (US)

(72) Inventor: Philip Arnold Ferolito, Sunnyvale, CA (US)

(21) Appl. No.: 14/795,869

(22) Filed: Jul. 9, 2015

Related U.S. Application Data
(60) Provisional application No. 61/998,798, filed on Jul. 9, 2014.
(60) Provisional application No. 62/042,728, filed on Aug. 27, 2014.

(51) Int. Cl. A61N 5/06

(52) U.S. Cl. CPC A61N 5/0616; A61N 5/0618; A61N 2005/0626; A61N 2005/063; A61N 2005/0645; A61N 2005/0652; A61N 2005/0659; A61N 2005/0661; A61N 2005/0663; A61N 2005/067

(57) ABSTRACT

A wearable device for therapeutic irradiation of skin may comprise: a light source optically coupled to a light spreading sheet and electrically coupled to a controller configured for controlling the intensity of light emitted from the light source and the duration of emission of light from the light source during a therapeutic session; a proximity sensor for detecting proximity of the light spreading sheet to the skin, the proximity sensor being attached to the light spreading sheet and electrically coupled to the controller; and a power source electrically coupled to the light source and the controller; wherein the controller is further configured to turn on, and keep turned on for the duration of the therapeutic session, the light source when the proximity sensor detects proximity of the light spreading sheet to the skin. In embodiments the light source may comprise an array of light emitting diodes attached to a substrate.

### CROSS-REFERENCE TO RELATED APPLICATIONS

This application claims the benefit of U.S. Provisional Application No. 61/998,798 filed Jul. 9, 2014 and U.S. Provisional Application No. 62/042,728 filed Aug. 27, 2014, both incorporated by reference in their entirety herein.

### FIELD OF THE INVENTION

The present invention relates generally to a wearable therapeutic light source and more specifically, although not exclusively, to a wearable ultraviolet, blue, red, near infrared, and/or infrared light source for treatments such as: healing of wounds, reduction of scars, stimulation of vitamin D synthesis, reduction of inflammation, regulation of immune response, resolution of pigmentation issues, and abatement of seasonal depression.

### BACKGROUND

There is a need for improved therapeutic light sources suitable for treatment of human patients, for enabling ultraviolet, blue, red, near infrared, and/or infrared light therapy for wound healing, scar reduction, vitamin D synthesis, inflammation reduction, regulation of immune response, resolution of skin pigmentation issues, seasonal depression abatement, etc.

### SUMMARY OF THE INVENTION

According to some embodiments, the present invention relates generally to a wearable therapeutic light source and more specifically, although not exclusively, to a wearable ultraviolet, blue, red, near infrared, and/or infrared light source for treatments such as: healing of wounds, reduction of scars, stimulation of vitamin D synthesis, reduction of inflammation, regulation of immune response, resolution of pigmentation issues, and abatement of seasonal depression.

According to some embodiments, a wearable device for therapeutic irradiation of skin may comprise: a light source; a light spreading sheet optically coupled to the light source, the light spreading sheet having a first surface and a second surface; a controller electrically coupled to the light source, the controller being configured for controlling the intensity of light emitted from the light source and the duration of emission of light from the light source during a therapeutic session; a proximity sensor for detecting proximity of the light spreading sheet to skin, the proximity sensor being attached to at least one of the first surface and the second surface of the light spreading sheet, the proximity sensor being electrically coupled to the controller; and a power source electrically coupled to the light source and the controller; wherein the controller is further configured to turn on, and keep turned on for the duration of the therapeutic session, the light source when the proximity sensor detects proximity of the light spreading sheet to the skin.

According to some embodiments, a wearable device for therapeutic irradiation of skin may comprise: a substrate, the substrate having a first surface and a second surface; a light source comprising an array of light emitting diodes (LEDs) attached to the first surface of the substrate; a controller electrically coupled to the light source, the controller being configured for controlling the intensity of light emitted from the light source and the duration of emission of light from the light source during a therapeutic session; a proximity sensor for detecting proximity of the substrate to skin, the proximity sensor being attached to at least one of the first surface and the second surface of the substrate, the proximity sensor being electrically coupled to the controller; and a power source electrically coupled to the light source and the controller; wherein the controller is further configured to turn on, and keep turned on for the duration of the therapeutic session, the light source when the proximity sensor detects proximity of the light spreading sheet to the skin.

According to some embodiments, a method of irradiating a patient's skin with a wearable device may comprise: providing a wearable device, the wearable device comprising a substrate, the substrate having a first surface and a second surface, a light source attached to the substrate, a controller electrically coupled to the light source, the controller being configured for controlling the intensity of light emitted from the light source and the duration of emission of light from the light source during a therapeutic session, a proximity sensor for detecting proximity of the substrate to the patient's skin, the proximity sensor being attached to at least one of the first surface and the second surface of the substrate, the proximity sensor being electrically coupled to the controller, and a power source electrically coupled to the light source and the controller; wherein the controller is further configured to only turn on, and keep turned on, the light source when the proximity sensor detects proximity of the substrate to the patient's skin; placing the wearable device in proximity to the patient's skin; detecting proximity of the light spreading sheet to the patient's skin by the controller; and on detecting proximity, turning on the light source by the controller for a prescribed time.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIGS. 1 & 2 are schematic representations of different embodiments of a wearable device for therapeutic irradiation of skin, according to the present invention;

FIGS. 3A & 3B show exploded and bottom views of a representation of a wearable device for therapeutic irradiation of skin, according to some embodiments of the present invention;

FIG. 4 shows examples of different locations for affixing the wearable device of FIGS. 3A & 3B, according to some embodiments of the present invention;

FIGS. 5A, 5B & 5C show exploded, top and bottom views of a representation of a wearable device for therapeutic irradiation of skin configured as a patch, according to some embodiments of the present invention;

FIG. 6 shows an example of a location for affixing the wearable device of FIGS. 5A, 5B & 5C, according to some embodiments of the present invention;

FIG. 7 is an action spectrum for the synthesis of vitamin D hormone in humans due to exposure of the skin to light;

FIG. 8 is a plot of percent conversion of 7-DHC to preD3 as a function of time for human skin exposed to different wavelengths of light;

FIG. 9 is a plot of MEDs as a function of wavelength for human skin exposure;

FIG. 10 is a plot of percent conversion to vitamin D as a function of time for different skin types;

FIGS. 11A & 11B show top and bottom views of a representation of a wearable device for therapeutic irradiation of skin including a sensor for measuring the amount of ambient light falling within the action spectrum for the therapeutic process, according to some embodiments of the present invention;

FIG. 12 shows an example of a location for affixing the wearable device of FIGS. 11A & 11B, according to some embodiments of the present invention;

FIG. 13 illustrates the conical nature of light dispersion from an LED;

FIG. 14 illustrates the uniform nature of illumination from an edge illuminated light spreading sheet, according to some embodiments of the present invention;

FIG. 15 shows a representation of an edge illuminated light spreading sheet comprising a material with embedded particles, according to some embodiments of the present invention;

FIG. 16 shows a representation of an edge illuminated light spreading sheet comprising a material with holes, according to some embodiments of the present invention;

FIG. 17 shows a representation of an edge illuminated light spreading sheet comprising a ribbon of fibers, according to some embodiments of the present invention;

FIG. 18 shows a representation of an edge illuminated light spreading sheet comprising a woven fabric, according to some embodiments of the present invention;

FIGS. 19A, 19B & 19C show exploded, top and side views, respectively, of a representation of a wearable device comprising an edge illuminated light spreading sheet, according to some embodiments of the present invention;

FIG. 20 shows an exploded view of a representation of a flexible wearable device comprising an edge illuminated light spreading sheet, according to some embodiments of the present invention;

FIGS. 21A & 21B show bottom and side views of a representation of a segmented wearable device, according to some embodiments of the present invention;

FIG. 22 shows a bottom view of a further embodiment of a segmented wearable device, according to the present invention;

FIG. 23 shows a representation of a wearable device comprising a printed opical diffuser, according to some embodiments of the present invention;

FIGS. 24A & 24B show bottom and side views of a representation of a wearable device comprising an array of LEDs on a substrate, according to some embodiments of the present invention;

FIG. 25 shows a bottom view of a representation of a wearable device comprising offset arrays of LEDs on a substrate, according to some embodiments of the present invention;

FIGS. 26A & 26B show bottom and cross-sectional views of a wearable device comprising an array of LEDs on a flexible substrate, according to some embodiments of the present invention;

FIGS. 27 & 28 are plots showing the angular distribution of illumination flux for an LED with a regular dome lens and an LED with a dome lens coated with a filter, respectively, according to some embodiments of the present invention;

FIGS. 29A & 29B show cross-sectional and top views, respectively, of an LED with a regular dome lens; and

FIGS. 30A & 30B show cross-sectional and top views, respectively, of an LED with a dome lens coated with a filter, according to some embodiments of the present invention.

### DETAILED DESCRIPTION

Embodiments of the present invention will now be described in detail with reference to the drawings, which are provided as illustrative examples of the invention so as to enable those skilled in the art to practice the invention. Notably, the figures and examples below are not meant to limit the scope of the present invention to a single embodiment, but other embodiments are possible by way of interchange of some or all of the described or illustrated elements. Moreover, where certain elements of the present invention can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

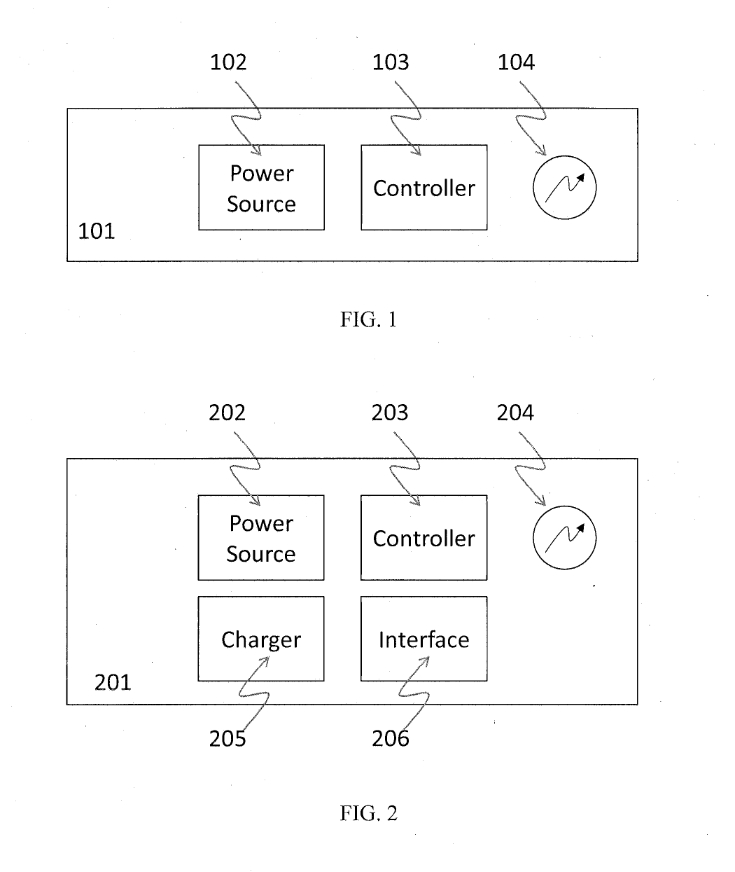

FIG. 1 shows a schematic of a first embodiment of a wearable device for therapeutic irradiation of skin (101) comprising a power source (102), such as a battery, a rechargeable battery, a supercapacitor, a mini fuel cell, etc., a controller (103) and at least one light source (104).

FIG. 2 shows a schematic of a second embodiment of a wearable device for therapeutic irradiation of skin (201) comprising a power source (202), controller (203), at least one light source (204), a charger or charger interface (205), and a control interface (206) capable of modifying the device function as described in more detail below.

As described above, the wearable device may contain a power source such as a battery, a rechargeable battery, a supercapacitor, a mini fuel cell, etc. for local energy storage. The battery may be replaceable or the battery and/or capacitor may be charged by any method including, but not limited to, using a wireless charger, a wired/cabled charger, and/or kinematic charging (harnessing energy from the motion of the wearer).

The wearable device may contain one or more light emitters providing light toward the wearer's skin. These light emitters can be narrow or broad spectrum, and may in embodiments include light emitters with output at different frequencies. Furthermore, in embodiments the device may contain one or more other light sources not directed toward the skin and unrelated to the therapeutic light source(s)—these light source(s) could provide the wearer with an indication of the status of the device, including but not limited to on/off status of the therapeutic light source(s), charge status, and other warnings or indicators.

The wearable device may contain a control interface allowing programmatic settings of the wearable device. The control interface can be of any type including but not limited to wireless, wired, electrical, or optical. The control interface may allow, but is not limited to, setting one or more light frequencies to emit, setting one or more power levels for the light source, setting a schedule of light generation. The schedule may for example be: a periodic schedule, such as daily, weekly, monthly or yearly, for example: turn on for 10 minutes every day from 8 am to 8:10 am; or turn on for 1 minute at the start of each hour from 9 am to 5 pm; or a non-periodic schedule, such as operating on Monday between 8 am to 8:10 am, Tuesday between 7:00 pm to 7:30 pm, and so forth.

The programmability of the wearable device may also allow for personalized adjustments to exposure duration and/or intensity allowing for individual needs to be accounted for. Adjustable pulse width modulated control can be used to optimize efficiency and therapeutic effects. Furthermore, the programmability may allow for a periodic or flexible programmable schedule, for example, based on time of day.

The wearable device may contain an interface allowing information gathered by the device to be downloaded to other devices including but not limited to computers and/or smart phones. Information obtained from this interface may include but is not limited to, device charge status, actual emitted light power and duration, or any other state the device may have. Additionally downloaded information can be real time or recorded sensor data including but not limited to skin pigmentation and skin contact status from one or more skin sensors.

The wearable device may contain a diffuser, or other light spreading configuration of materials, to increase the exposed area of skin and thus reduce the exposure power per unit area. These diffusers include but are not limited to one or more of lenses, fibers, light pipes and/or reflective surfaces. In general practice the diffusing element would be optimized to minimize attenuation at wavelengths produced by the light emitters, or the desirable part(s) of the emitted spectrum from the light emitters. On surfaces not adjacent to the skin, the diffuser may also include coatings, reflective at the emitter wavelengths, to both boost the efficiency of the system by returning light back toward the skin and to prevent leakage of light which might lead to exposure of unintended surfaces of both people and objects.

The wearable device may contain one or more detectors able to measure the presence of, or intensity of, backscattered light. Data from this sensor, or sensors, can be used in a variety of ways, including but not limited to automatic adjustments in light emitter intensity and/or duration for safety or compensation for variations in skin, such as skin pigmentation. Pigmentation can be measured by a variety of methods including but not limited to measuring the reflected light from sources of specific wavelengths or taking a picture of a small portion of the skin using a broad spectrum or white light source. A simple method of skin pigmentation detection involves taking the equivalent of a small photograph of a section of skin using a CMOS (complimentary metal-oxide semiconductor) or CCD (charge-coupled device) sensor and a visible light source such as a white LED. The light source for the sensor can be local to each sensor or shared by coupling it in to the diffuser. Skin pigmentation can then be determined using software analysis of the data from the sensor. A small known color target can be included in the picture taken to increase accuracy by having a known color to compare. Ideally several sensors would be located at intervals across the device to reduce the probability of a sensor aligning to a skin feature including but not limited to a scar, pimple, mole, or hair, and creating a false pigmentation reading. Multiple sensors and/or sensors capable of averaging or viewing a wider area of skin provide protection from single point sampling error. Finally, the controller might sample these sensors at the start of a session, or periodically during treatment. Sensors may also detect the amplitude of emitted light and send to the controller as feedback for adjusting the amplitude to maintain a constant and predictable level; compensating for factors including but not limited to variations in the light emitters, aging effects (dimming) of the light emitters, variations of transparency of the diffusive materials, and degradation of transparency of the diffusive materials. Ideally, optical to electrical sensors capable of measuring the illumination of each therapeutic wavelength are located both at the farthest distances from the light source as well as strategic intermediate locations within or along the edge of the wearable device. These sensors measure the illumination at specific locations, providing feedback data to the controller. The controller then adjusts the illumination of the corresponding light source (increase or decrease) to provide a predicable amount of delivered light. If multiple sensors for a therapeutic wavelength are present and multiple light sources, with independent control are present, the controller can utilize topographical information about the light source and sensor physical locations to adjust the light sources to increase the uniformity of exposure delivered to the skin. The controller may sample these sensors at the start of a therapeutic session, at periodic intervals during a session, or continuously. A separate calibration session might be used to perform adjustments of the light sources while not in use. Finally the controller may log information regarding adjustments in a non-volatile memory for future use and diagnostics.

The wearable device can be worn in many ways including but not limited to a blanket, band or cuff (worn on the wrist, arm, leg, or ankle), a ring worn on a finger or toe, a patch directly affixed to any skin, a sleeve or pouch held proximate to a portion of skin anywhere on the body or an item of clothing. The wearable device can be worn in close proximity to the skin and in embodiments may conform to the skin of whatever body part the wearable device is attached to; herein in embodiments “close proximity to” and “conformal to” are within 2.5 cm.

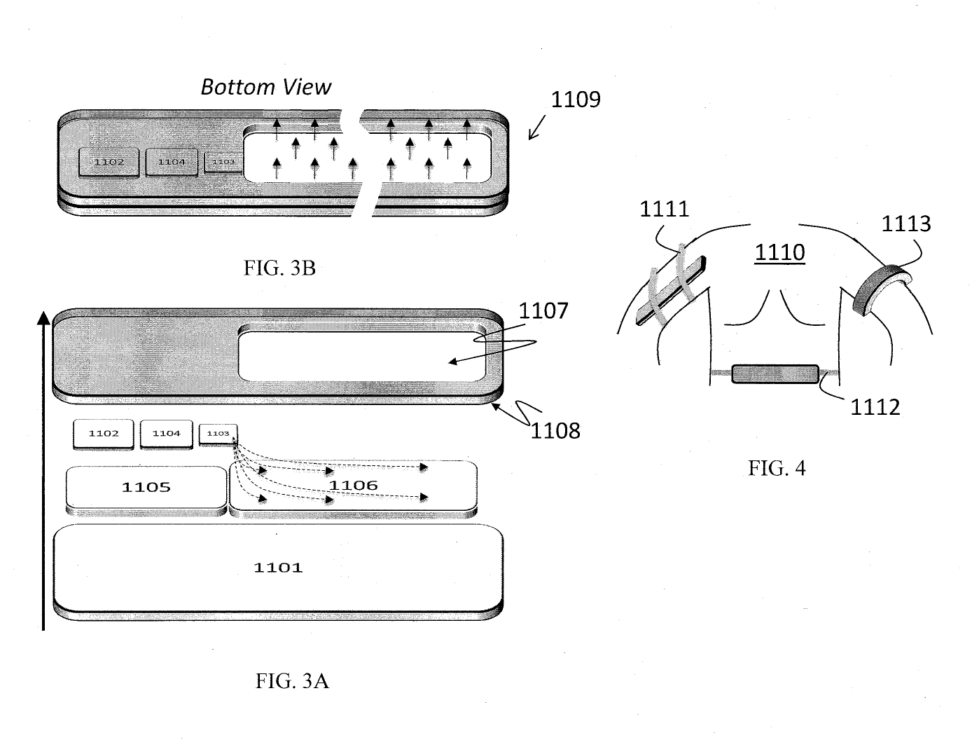

FIGS. 3A, 3B & 4 show a wearable device (1109) according to some embodiments, comprising a protective cover (1101), printed circuit board (1105), light distribution material (1106), battery and/or capacitor (1102), controller (1104), and one or more LEDs (1103), cover panel (1108) with aperture/opening (1107). The controller (1104) may also have a wireless interface for acquisition of electronic data such as skin type, any sort of exposure profile such as up to a maximum permitted exposure and/or monitoring. The wearable device if rigid or semi-rigid may be affixed to the wearer (1110) by straps (1111) or part of a belt or garment (1112). If flexible the wearable device may be worn as a wrap or band (1113). The device is envisioned to be worn next to the skin anywhere from the neck down, including but not limited to back, front or side of torso and any portion of arms or legs. Note the vertical arrows in FIG. 3B which indicate light emission from the wearable device, and also the dashed arrows in FIG. 3A which indicate the illumination of the light distribution material by the one or more LEDs.

Potential uses of the wearable devices of the present disclosure include but are not limited to long term increase, stabilization, and maintenance of serum vitamin D, 25(OH)D (25-hydroxyvitamin D) levels and co-dependent hormone levels such as parathyroid hormone (PTH). Another potential use of this device is continuous or intermittent exposure to light wavelengths stimulating the production of nitric oxide having the therapeutic effect of lowering blood pressure, for example by exposure to certain IR (infra-red) wavelengths and/or certain UV (ultraviolet) wavelengths. Further uses and corresponding therapeutic wavelengths are discussed below.

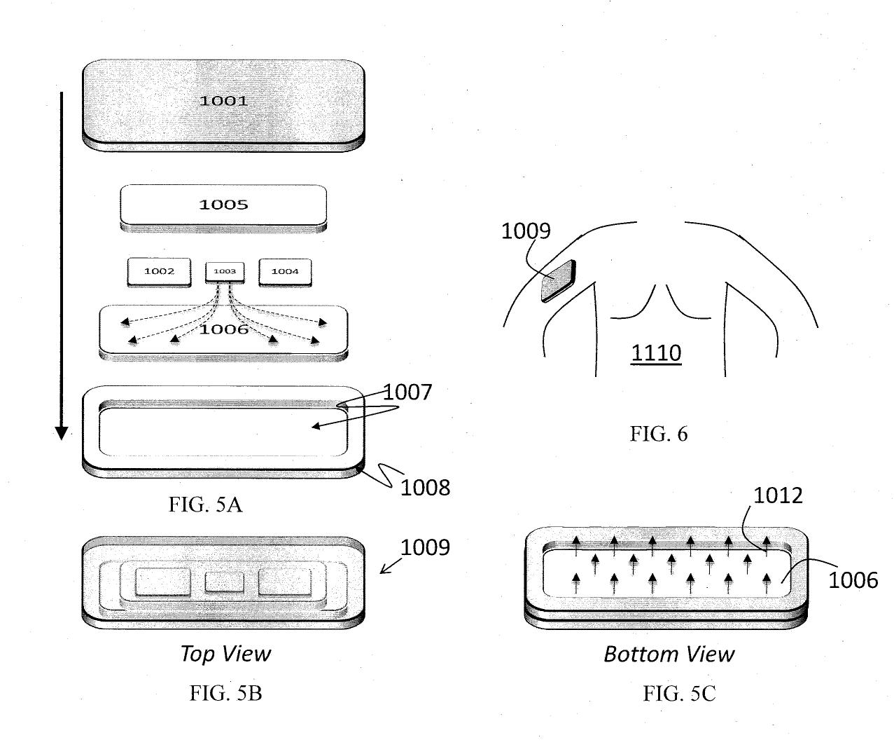

FIGS. 5A, 5B, 5C & 6 show a basic wearable device configured as a patch (1009). The device includes a cover (1001), printed circuit board (1005), battery or capacitor (1002), controller (1004), one or more light emitting LEDs (1003), a light transport material (1006), and an adhesive ring (1008) with aperture/opening (1007). The device may also contain a wireless interface for configuration and/or monitoring. The device is envisioned to be worn next to the skin, as described above, for a period of time sufficient to provide a therapeutic result. Note the vertical arrows 1012 in FIG. 5C which indicate the light emission from the wearable device, and also the dashed arrows in FIG. 5A which indicate the illumination of the light distribution material by the one or more LEDs. The wearable device of FIGS. 5A, 5B, 5C & 6 may be used for various fixed duration applications, including but not limited to accelerating wound closure, limiting bacterial growth, and reducing scaring.

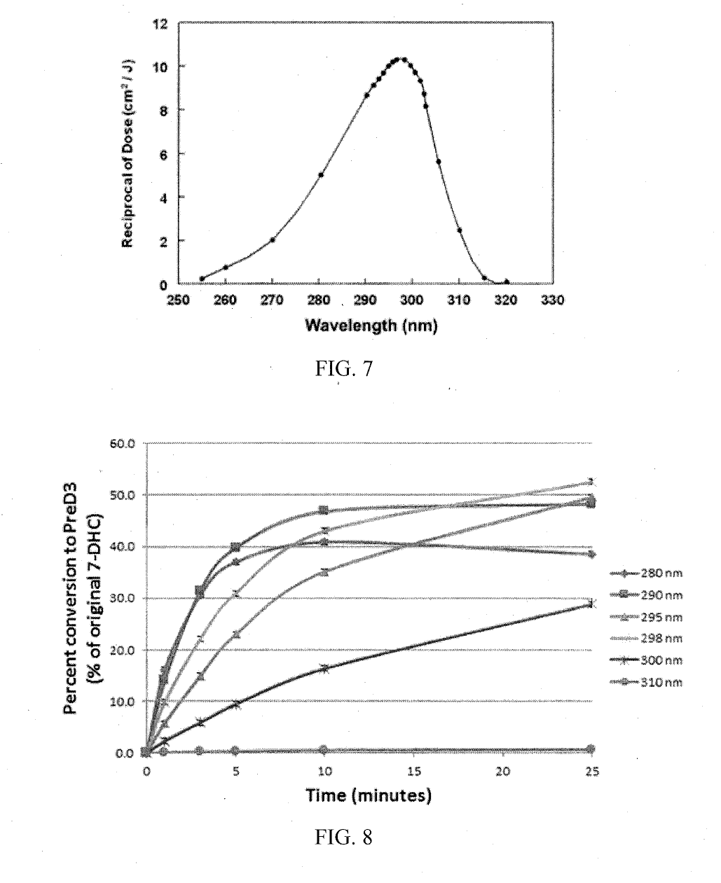

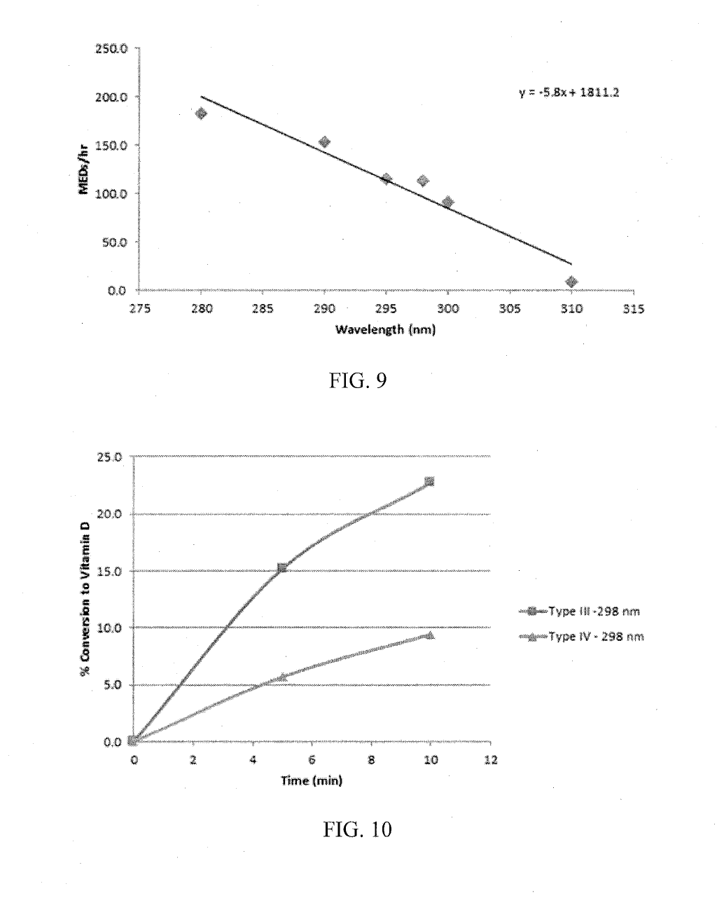

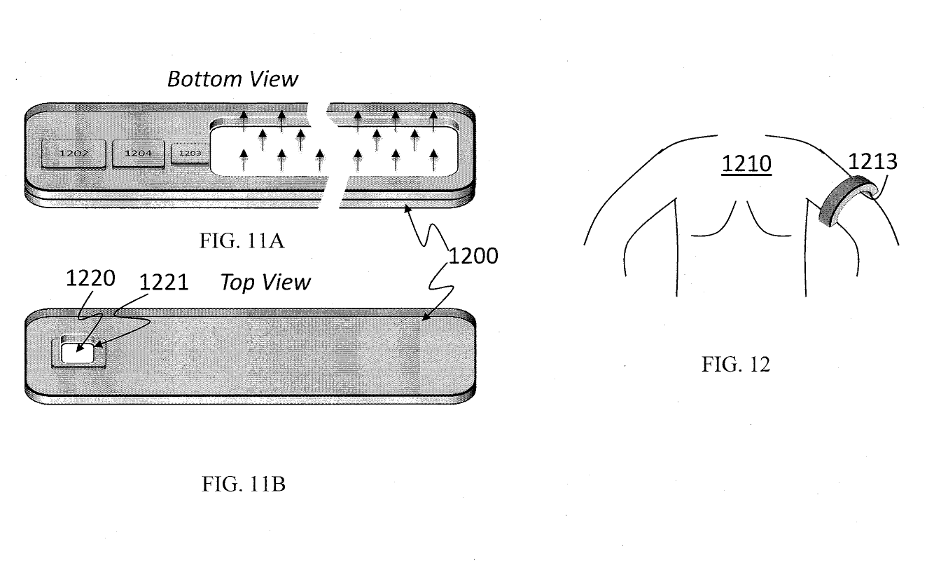

Synthesis of vitamin D hormone in humans begins with skin exposure to light falling within a wavelength range known as the action-spectrum. The action-spectrum is currently documented to span from 260 nm to 315 nm with a maximum conversion rate in the range of 295 nm to 300 nm. See FIG. 7 where the dependent axis shows a relative conversion rate of 7-Dehydrocholesterol (7DHC) to pre-vitamin D (preD) so that 298 nm light provides ten times the conversion rate of 310 nm light. FIG. 8 shows the percent conversion of 7-DHC to preD3 during continuous exposure using an LED light source with central spectral power as indicated in the figure and FWHM (Full Width Half Maximum) of around 10 nm—this demonstrates the ineffectiveness of wavelengths such as 310 nm for this conversion and highlights the effectiveness of wavelength near 295 nm. The effectiveness of a device to create preD needs to take in to account the CEI (Commission Internationale de l'Eclairage) spectrum weighting for UV exposure to skin. Wavelengths below 400 nm are weighted exponentially (much higher as the wavelength gets shorter) and the area under the spectrum is summed using this weighting to determine the skin exposure. FIG. 9 shows MEDs (Minimal Erythema Dose) as a function of wavelength, which reflects the weighting of the CIE—it is seen from the figure that as the wavelength reduces the contribution to MED is increased. When the skin exposure exceeds 1 MED exposure needs to be stopped. For example; a light source of 320 nm can expose up to the 1 MED limit and produce no PreD; a light source of 290 nm would reach the 1 MED limit long before a light source of 298 nm and have produced less preD. FIG. 10 shows the percentage conversion, using the preferred LED light source centered around 298 nm wavelength, to vitamin D as measured in human skin for skin of type III and type IV as measured on the Fitzpatrick scale (corresponding to light brown and medium brown, respectively). Clearly, significant adjustments must be made during light therapy for different skin types.

As wavelengths of light below 320 nm are described in the art as known carcinogens minimizing the intensity and duration of exposure not only improves battery life for a wearable device but also limits potential tissue and cellular damage. For reference, sunlight contains substantial energy from 280 nm and longer wavelength but the earth's atmosphere scatters, reflects and absorbs most of the light with wavelengths less than 315 nm.

FIGS. 11A, 11B & 12 show a further embodiment of a wearable device (1200) worn on the body (1210) at a location (1213) exposed to external UV light. Note the vertical arrows in FIG. 11A which indicate light emission from the wearable device. The device has a hole(s) (1220) to allow external light to fall upon a sensor (1221). The light may be allowed to directly fall upon the sensor (as shown) with protective covering over the sensor, or a light-pipe or similar structure could be used to guide light from one or more openings to the sensor. Any external illumination, from artificial or natural sources, with wavelength(s) falling in the action spectrum, suggest that other portions of the body are likely converting 7-DHC in the skin and this data can be used by a controller 1204 to adjust the UV light dose and delivery provided by the wearable device 1200. This can be further extended to any of the therapeutic wavelengths, saving device power when the ambient conditions are providing adequate energy at the desired wavelengths.

Using the data gathered regarding the external light spectrum, specifically the intensity and duration of ambient UV light falling within the action-spectrum, a processor in or coupled with the wearable device can modify the UV light exposure method to provide a correct dose of vitamin D. For example, the UV light exposure method may skip illumination cycles when the recent cumulative ambient UV radiation, falling within the action spectrum, indicates no further conversion is required. A recent history of ambient light falling within the action spectrum can be used to reduce device power consumption, extend battery life, and avoid unnecessary exposure of a patient's skin. The next exposure might be delayed if sufficient natural UVB exposure is occurring or has occurred.

An additional sensor can be added on the skin-facing side of the wearable device to measure light backscattered from the surface of the skin. This can be used to assess the skin color which can be provided as an input in embodiments of the methods of the present invention to make adjustments to intensity, duration and relaxation interval between UV light exposures. For example, the Fitzpatrick scale defines 6 different skin types spanning from fair (pale) to heavily-pigmented (dark). Other embodiments may utilize a scale with at least 3 skin types. In further embodiments a scale with more than 16 different skin types might be used. Pale skin color will reflect more light, while heavily pigmented skin will absorb more light. Darker skin types have a naturally higher resistance to UV damage and can tolerate longer or more intense exposure to UV light. Lighter skin colors react more quickly to a UV light source. Knowledge of skin color/type can be configured in the algorithm manually by the user, however the backscatter sensor allows adjustments to the exposure intensity and exposure duration to take in to account measured light reflections directly from the skin.

In the event that the backscatter sensor detects the light is below a specified threshold, the device can determine that it is not against the surface of the skin and disable the LEDs. This would prevent the device from actively driving the UV LEDs when the device itself is placed face-up on a surface or being held so that UV illumination could escape in to a room. This is primarily a safety feature, since UV illumination can cause eye damage, for example.

A capacitive skin sensor can be incorporated in to some embodiments of the device to detect when the device is actually being worn and worn correctly. Embodiments of the wearable devices described herein can use data from capacitive sensors to make closed-loop automatic adjustments to the UV dose, rate and delivery. For example, if the skin sensor indicates that the device is not being worn against the skin, the UV LEDs can remain un-illuminated saving both power and reducing any potentially harmful UV exposure.

Since various sensors (backscatter, capacitive skin proximity sensor, or ambient UVB sensors) can dynamically make adjustments to the exposure algorithm, the wearable device may maintain a log of how much actual exposure (or dose) was delivered to the wearer. This data is stored internal to the device and can be extracted via a wireless or wired connection to a smart phone, tablet or other computing device. This data can further be accumulated and presented to the wearer by any number of events or alerts. For example, the wearer who is wearing the device outside a shirt (instead of against the skin) would be alerted to the fact that the Capacitive Skin Sensor has forced the skipping of exposures. Another example would be to alert the wearer of the total UVB dose delivered and reasons why exposures were skipped, including but not limited to low-power, and worn incorrectly (as determined by the backscatter or Skin sensor).

In an embodiment, a row, or several rows, of LEDs forming an array of individual LEDs may be used as the UV light source. LED “ON” times may be adjusted so that the peak current draw from the power supply is reduced—in an example of using 4 LEDs, the current draw from the power supply need not be more than a device with a single LED if only one diode is activated at a time. A simple row of 4 LEDs is one example, although many alternative patterns could be used which are in essence the same principle. As the number of diodes increases it may be desirable to have 2 or more LEDs on at the same time.

UV LEDs efficiency (power out/power in) is improving annually, however, the efficiency of these devices is still orders of magnitude below that of visible light and IR diodes; furthermore, the component cost of UV LEDs, although dropping, is high—three orders of magnitude higher than for IR or visible LEDs. For greater time between battery recharging and for lower cost wearable devices, some embodiments may use a small number of LEDs, as few as one, and an edge illuminated light spreader to project the light across the targeted expanse of skin.

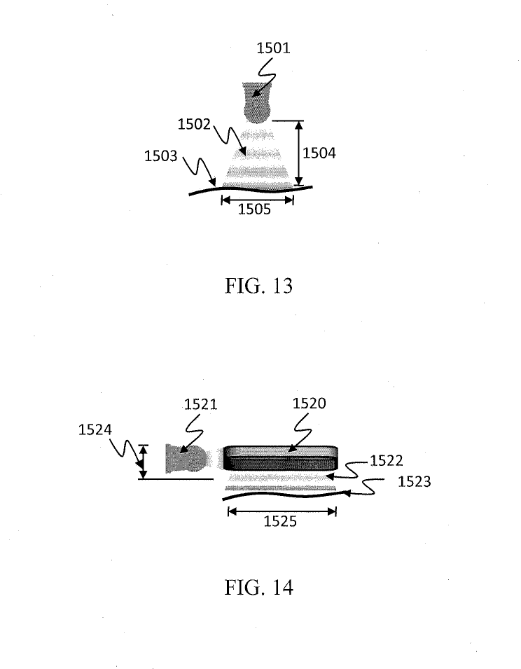

As seen in FIG. 13 a UV LED (1501) emits light (1502) toward the skin (1503). Due to the conical nature of the light spreading from the LED source, the distance (1504) from the skin (1503) determines the total area of skin illuminated by the light source. Adding a lens to the LED or simple diffuser/lens between the LED and the skin can increase the dispersion angle but there still remains a strong relationship between the distance the LED is from the skin (1504) and the total area of skin covered by the light (1505). In contrast, as seen in FIG. 14, a similar or same UV LED (1521) emitting light in to an edge illuminated material (1520) with good optical properties in the UVB range allows transport of the UV light (1522) across an arbitrary extent (1525) of skin (1523) while maintaining a fixed device thickness (1524). The extent of skin over which the UV light may be distributed is limited only by the output power of the LED, optical properties of the diffuser (1520) and desired intensity at the surface of the skin. For example: if a single light source is used, it has to be held a good distance away from the subject to illuminate a wide area of skin, where as an edge illuminated diffuser could allow the material to be held in close proximity (touching) the skin and be only a few millimeters thick. In other embodiments a thin blanket may use many LEDs arranged in an array, this provides the ‘thinness’ but will cost substantially more due to the large number of LEDs required to cover a large area. If more intensity is required than can be provided by a single UV LED, a 2nd or more LED(s) may be added, sharing the same light spreading material (1520) or using a 2nd (or more) piece of material in parallel.

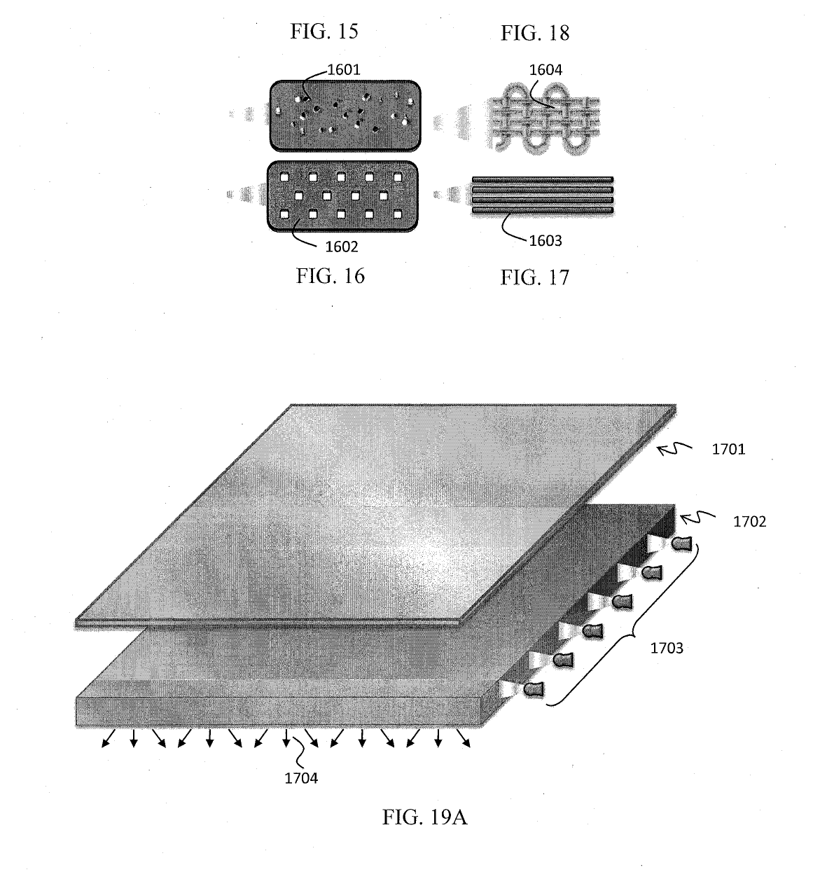

Many potential and equivalent structures potentially exist for the realization of the edge illuminated light spreader. In an embodiment, the edge illuminated light spreader structure would be strong and flexible. Options for construction of the light spreader include a single monolithic waveguide, a ribbon of fibers (1603), a braid of fiber, or folded optics waveguide, or woven material like a fabric (1604). The light spreader could be constructed of, for example, a homogeneous material, layers of different materials, a piece of faceted material, material with embedded particles (1601), a material with holes (1602), graded indexed materials or nanostructures. See FIGS. 15-18, which show representations of edge illumination of examples of a light spreading sheets. Various methods of creating a bulk diffuser all starting with at least one material which is very transparent to the therapeutic light, for example UVB light. The material would ideally be flexible. An advantage of fabric style materials is flexibility and the ability to directly weave the material (such as an optical fiber) in to garments or other wearable clothes and accessories. A desirable property of any style of light spreader for this application is that it has low loss for light across the target spectrum, for vitamin-D this is 290-320 nm. In an embodiment, the material due to impurities (such as added particles (1601)) or physical construction (by adding cuts, holes, or variation in index of refraction along the material) will cause light to exit the material along at least 1 face. (Materials with light exiting the side are generally referred to as lossy waveguides or lossy fibers.)

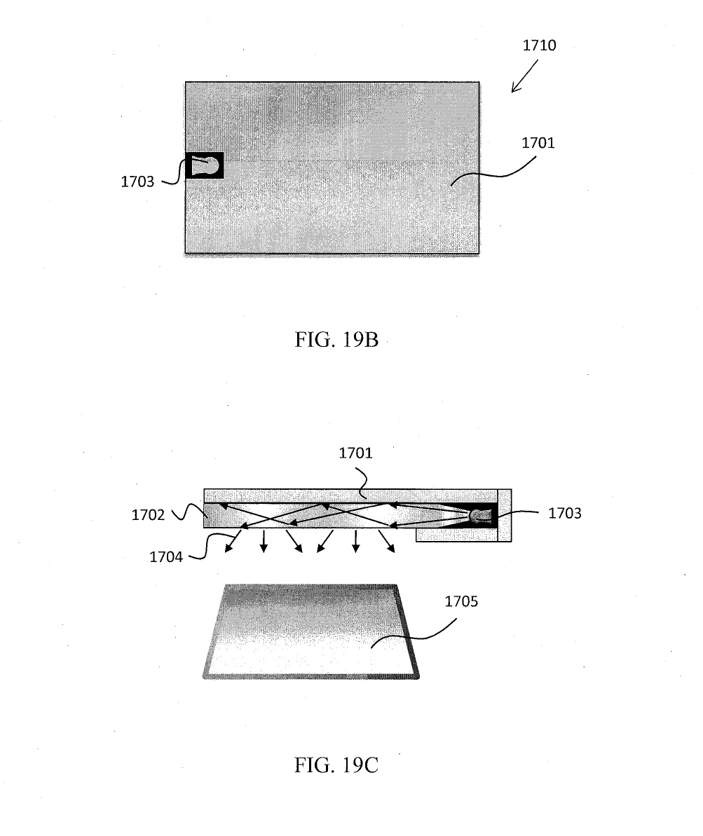

A highly flexible light spreader would allow conformity to the body on which it is affixed, providing a low profile and very predictable exposure over a wide range of dimensions. A flexible but slightly rigid light spreader allows for partial conformity to the body, limiting the size of the device or limiting the target body areas to broad ranges with minimal contours. A rigid inflexible light spreader would limit the exposure area of the device. Flexible/foldable light spreaders are discussed below for use as wearable blankets, for example. Further details are provided below with reference to FIGS. 19A, 19B, 19C, 20, 21A, 21B & 22.

The wearable device may contain one or more visible light LED(s). One or more of these LEDs can be illuminated while the UV LED(s) are active. This provides a visible indication that the device is active and working. If part of the visible light is also directed through the light diffuser, the visible indicator can be viewed as a safety feature.

The wearable devices described herein can be combined with other wearable technologies including but not limited to a pedometer or other bio or motion sensors.

Adding a RTC (real time clock) would allow the controller of the wearable therapeutic light sources to account for time of day and day of year adjustments to the profile. Adding a method for obtaining global positioning information, statically configured or dynamically obtained from a GPS device, allows the exposure profile to be adjusted based on seasonal variations of ambient light exposure. For example, in wearable devices targeting vitamin-D deficiency required UV exposures would be less for equatorial locations or summer months in moderately northern locations.

FIGS. 19A, 19B & 19C show different views of a wearable device according to some embodiments. FIG. 19A is an exploded representation of the wearable device comprising a UV reflective covering (1701), a UV waveguide (1702) with lossy lower surface (1704) and one or more LEDs (1703). FIG. 19B is a top view (side of wearable device facing away from wearer's skin) of the edge illuminated panel (1710). FIG. 19C shows a cross-section of the wearable device with a perspective view of a protective panel (1705), which may be comprised of UV transparent or UV diffuse material, which may, in certain embodiments be affixed to the lossy lower surface (1704) of the UV waveguide (1702).

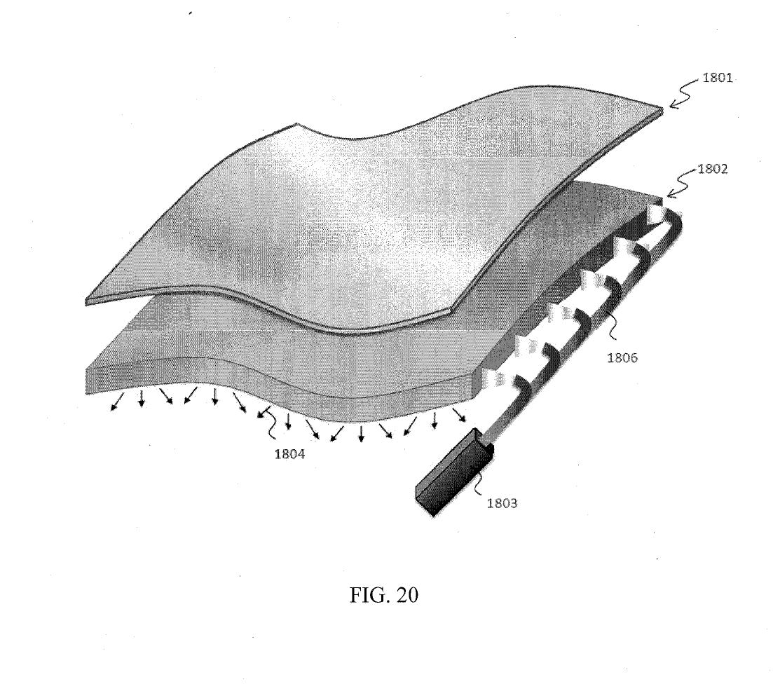

FIG. 20 illustrates a flexible/conforming version of the wearable device comprising UV reflective covering (1801), LEDS (1803) connecting to diffusive material (1802) with optical fibers (1806).

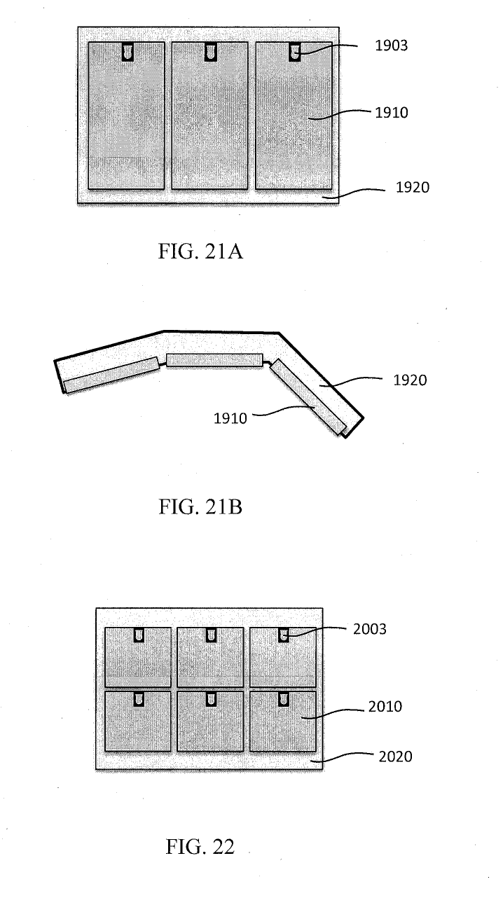

FIGS. 21A & 21B show a representation of a linear segmented blanket 1920 comprising edge illuminated panels 1910, each panel having one or more LEDs 1903. The panels may be attached with flexible hinges/joints. Small panels may be rigid but overall the blanket may be conforming to the body of a wearer. The blanket may be formed of a sturdy material and including the required wiring to control the LEDs on the segmented panels. This wiring may be a physical cable, printed circuits, etc. There is at least one LED per panel for the range of wavelengths desired. Each panel may also contain a sensor to detect the power level of each LED so that the lifetime dimming of the devices may be compensate for and LED failures may be detected. A second redundant LED per desired wavelength range may be added to provide further redundancy.

FIG. 22 shows a segmented blanket 2020 comprising an array of panels 2010, each panel having one or more LEDs 1903.

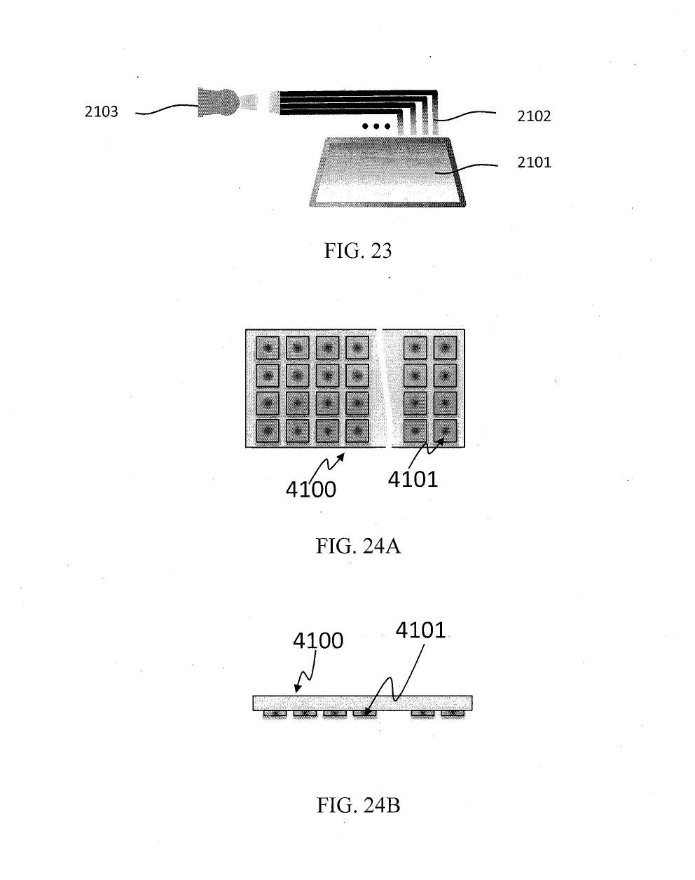

In further embodiments a printed diffuser may be used in a wearable device. FIG. 23 shows such a printed optical diffuser 2101 coupled by a series of waveguides 2102 to one or more LEDs 2103. One method of distributing light to a wide area is to use a bundle of fibers that all originate at the source (LED) and are spread out to discrete points of varying lengths to different portions of the wearable device. The fibers themselves can be lossy in embodiments or loss-less in other embodiments. Instead of discrete fibers, printed waveguides may be used. Printing might be accomplished by using ink-jet printer technology, or similar, to put down a pattern of waveguides composed of one or more materials with the desired optical properties (refractive index, etc.) that can be cured to stable solid form after printing. Curing is typically accomplished by exposure to certain wavelengths of light or through the application of heat and time depending on the materials being used. Alternatively the printing process could use a traditional screen printing process.

In yet further embodiments a wearable device may comprise an array of LEDs affixed to a substrate, and in embodiments the LEDs may be printed LEDs. FIGS. 24A & 24B show (printed) LEDs 4101 in an array on a substrate 4100. Herein the aerial density of the array refers to the number of LEDs on a unit area of substrate, and thus reflects the spacing between LEDs. The substrate material may include Polyethleneterephthalate (PET) foil Polyethylenenaphthalate (PEN) foil or Polyimide (PI) foil, for example. Additionally metal foils or laminates can be applied to add some durability and aid with heal dissipation, though only on a surface of the substrate not required to be transparent. The printed LEDs might be covered by a protective coating that is transmissive to the therapeutic wavelengths. Furthermore, a coating to disperse the LED light might be added on top of the printed LEDs, depending on the density of LEDs which can be achieved. (If the density of LEDs is high enough then the dispersion coating might not be required.)

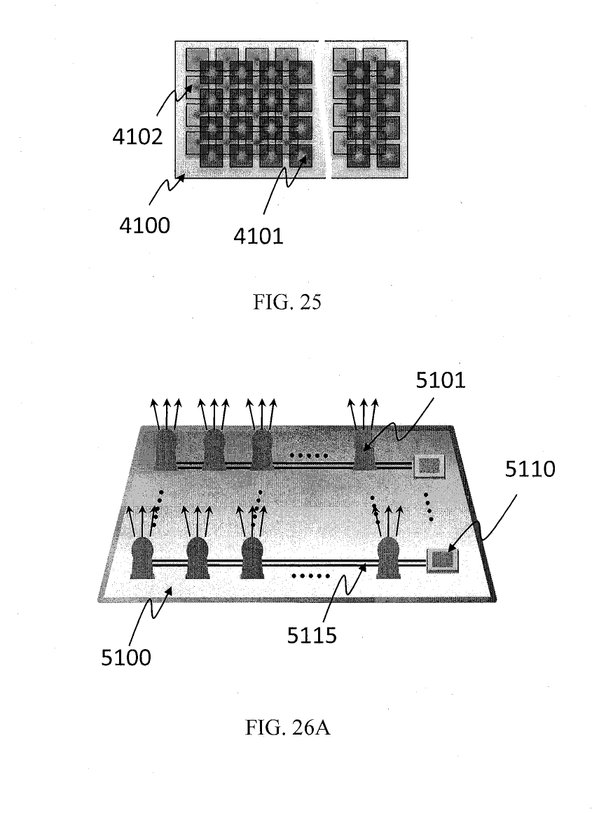

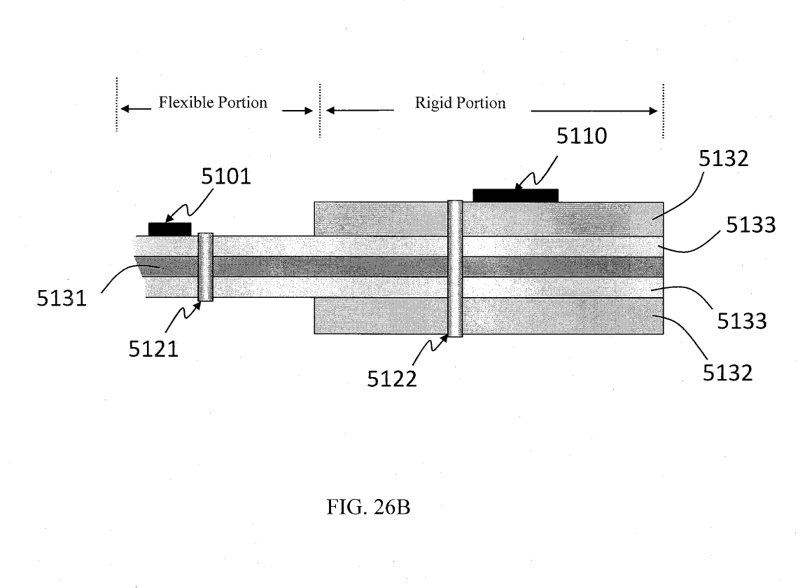

In some embodiments, as shown in FIGS. 26A & 26B diodes 5101 may be placed in an array on a (flexible) substrate 5100 so as to directly illuminate the target skin. The substrate may contain traces 5115 to electrically connect the diodes to one or more controllers 5110 or discrete wires can be used. The substrate may in embodiments comprise a polyimide layer 5131, adhesive layers 5133 and rigid layers 5132, where the rigid layers may be limited to a first portion of the substrate to form a rigid portion on which controllers are affixed, and the remaining portion of the substrate may be flexible to enable conformal application to a part of the human body as a wearable device. Vias 5121 and 5122 allow connection of the surface mounted controller 5110 to internal metal traces (such as in printed circuit boards, not shown in figure) and surface mounted LEDs 5101 to internal metal traces—the internal metal traces connecting all components as desired for operation of the device. Furthermore, in embodiments the vias may connect to metal traces or wiring on the back side of the substrate. The diodes can be arranged so as to emit light away from the substrate, as indicated by arrows in the figures, although other embodiments may comprise diodes arranged to emit light into a substrate that acts as a light spreading sheet. The diodes can be arranged as a 2 dimensional array, with regular rows and columns or staggered rows and/or columns. The purpose of the array is to provide a relatively uniform illumination, so the arrangement should minimize the overlap of light from adjacent diodes unless the sum of the light from adjacent diodes is determined to be less than the peak elsewhere in the array. Diode groups, each comprised of diodes of the same target wavelength, can be arranged such that each wavelength group independently provides non-overlapping illumination or where there is overlap of emitted wavelengths the integrated emission is controlled to avoid exceeding the desired dose at all therapeutic wavelengths. Diode groups comprised of different wavelengths can be offset from the previous wavelength diode groups so as to allow each additional diode group to illuminate the same basic area as the original diode group, as shown in FIG. 25 where a first diode array 4101 is offset from a second diode group 4102 on a substrate 4100. Feedback sensors (not shown in the figure), may be situated along the edge of the substrate surface for detecting light at a therapeutic wavelength, the sensors being electrically coupled to the controller(s), wherein the controllers may adjust light intensity of the LED light source in response to input from the sensors.

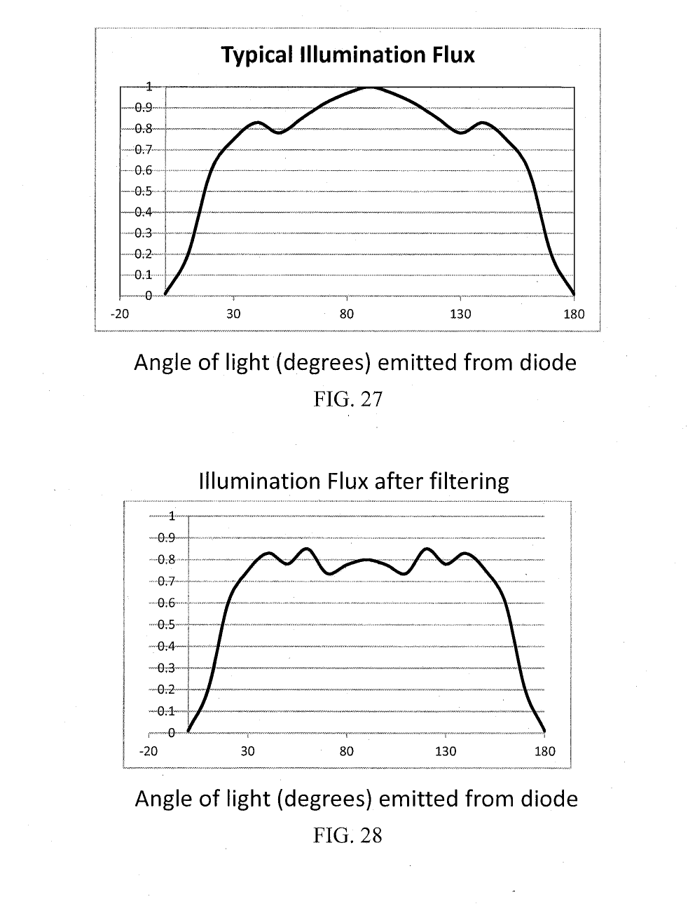

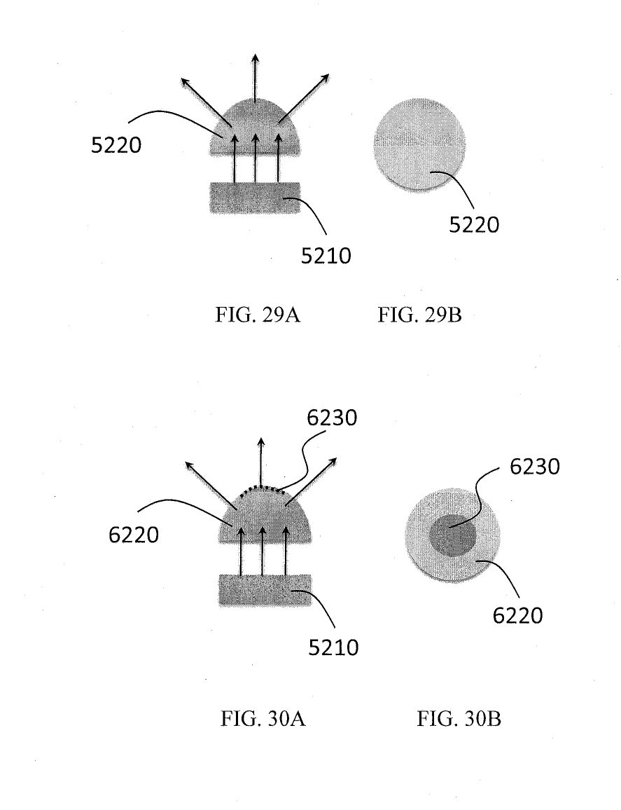

Diodes can have filters added to improve uniformity by reducing the illumination where the radiation pattern is highest (generally near the zenith of the dome lens or the center of the flat lens). Compare FIGS. 27 & 28 which show angular distributions of illumination flux for an LED 5210 with a regular dome lens 5220 and an LED with a dome lens 6220 and filter 6230, respectively; corresponding structures are shown in FIGS. 29A & 29B and 30A & 30B, respectively, where light emission from the LED and distribution through the dome lens is indicated by arrows. FIGS. 29A & 29B show cross-sectional and top views, respectively of an LED with a regular dome lens; and FIGS. 30A & 30B show cross-sectional and top views, respectively, of an LED with a dome lens coated with a filter. Due to the nature of most lenses, this filter coating most likely is restricted to a circular area centered on the lens, as shown in FIG. 30A. Multiple coatings may be applied, each consecutive one with progressively smaller radius to further reduce variation of emitted light by angle.

The diffusive material used in some embodiments herein may be characterized by: being flexible with a bend radius of less than 2 cm and a component lifetime of in excess of 4000 bending cycles over the wearable device lifetime; providing uniform diffusion of edge illuminated light sources; being compatible with primary light sources within the range of 290 nm to 310 nm; being able to diffuse and deliver 365 nm and 685 nm light as well; having approximate x-y dimensions of 21 cm×27 cm—in a single piece or multiple smaller strips; being non-reactive to human skin, or a protective layer may be utilized instead—interposed between the diffuser and the wearer's skin; 3 to 5 year service life with exposure to oils and dirt from human skin; having a thickness of greater than 350 μm for simple attachment of diodes; and being compatible with high volume manufacturing.

Furthermore, in embodiments illumination of the (typically rectangular) waveguide/diffuser may be from 2, 3 or 4 sides. When the wearable device/blanket is fabricated using multiple diodes, the diodes can be of the same wavelength range allowing mixing of the light and the controller can then compensate for wavelength or intensity variations of individual diodes. One or more sensors can be included to allow the control circuit to adjust exposure time and LED driving intensity to compensate for lifetime dimming of the LEDs. One or more sensors can be included to allow the control circuit to adjust the intensity of each diode independently to compensate for material and diode variations and provide as uniform as possible light emitting from the front of the blanket. Redundancy can be achieved by including at least one additional diode of each wavelength range (color)—since this represents N+1 redundancy, and the cost overhead reduces as N becomes larger.

Furthermore, a transparent, disposable or cleanable sleeve may be used in embodiments to prevent contamination in a clinical setting where a wearable device/blanket is shared among many patients.

Furthermore, in embodiments a method for distinguishing two or more users of the same wearable device/blanket and storing and recalling a profile for each user, including configuration information such as skin pigmentation and targeted therapy goals, may be used. This method can be implemented by using an indexed slider or knob or digital equivalent built into the wearable device/blanket for selecting a user by user number or enumerated tags such as a user name, etc.

Furthermore, in embodiments information may be collected and recorded for each blanket, include the following: recording the identification of the practitioner who initiated the treatment, such as a nurse or physician, which may include entering the practitioner ID or scanning the practitioner's badge or other means of identification; capturing the patient identification, time or day, date, and duration of treatment; recording a code for the location on the patient the blanket is to be applied. This data may be processed to show (perhaps graphically) a history of blanket applications to this patient that distinguishes the most recent from the earlier treatment sites. This can be done by using numbers, colors or another indicator and may be used by the practitioner to guide and select the next treatment site. Clearly, moving the blanket around to different sites on the body for treatment will reduce even further the potential for skin damage and optionally maximize photo product production. This information may be displayed on an external device (such as tablet or phone, or other) and/or an external device (such as a touch screen enabled tablet) to indicate the current treatment location choice.

Furthermore, in embodiments a user interface (on the wearable device/blanket or on an external device) may be used for display of dose readout and estimated IU (International Unit) equivalence. The information could be displayed using digital numbers, progress bars, or color coding.

Furthermore, in embodiments geographical location and the time of year may be used to provide adjustments to therapeutic UV doses needed. For example, when the wearer is closer to the equator the need for supplemental exposure is reduced, when the wearer is at higher elevation the wearer requires less supplemental exposure so long as the outdoor temperatures and cloud cover are conducive to exposing skin to sunlight, and when the wearer is at a location and time that indicates the winter season more supplemental UV exposure would be desired. Furthermore, the above may be enhanced by using live or near real-time UV index data to adjust exposure time/intensity.

Furthermore, in embodiments an LED odometer may be used to predict LED lifetime degradation by recording the duration and output of the LEDs. Diode dimming can be predicted based on the number of hours a diode has been active and the power levels at which the diode has been operated. This prediction is different for diodes made with different process technologies and there will most likely be variation between vendors and even generations of device from the same vendor. A table by vendor and diode revision may be compiled and used to adjust the predicted degradation of the light emitting devices. This LED odometer can be used to conserve energy in the power supply (e.g. batteries) by disabling the older diodes their useful life has expired. Furthermore, when UV light is present in sufficient quantity in the ambient environment the UV LEDs may be disabled to conserve energy and increase the time between UV LED replacement—for example: disable the UV diodes when the external UV index is high.

Furthermore, in embodiments a wireless interface may be used to load configurations into the wearable device/blanket controllers, or offload data or other monitoring

Furthermore, in embodiments a set of small round patches may be placed on moles or other sensitive skin structures within an area of the skin prior to application of the wearable device/blanket to the same area of skin. In individuals with specific skin features that need to avoid UV light sources, a small opaque patch can be applied to the skin which will remain affixed under the wearable device/blanket during treatment.

Furthermore, in embodiments a wearable device may comprise a controller/user interface that provides the user a mechanism to override the prescribed operating parameters of the wearable device within certain safe limits. For example, if the device determines that the exposure time should be N minutes with a power level of P due to the patients skin pigmentation (the DOSE can be considered as roughly N×P), then the patient can be provided a control allowing them to scale the treatment to S×N×P where S is a value between 0 and 1, for example 0.75. A setting of S=1.0 (the default) would correspond to the safe upper limit of exposure—this upper limit could be represented as 100% and the user could be provided with an input range from 20% to 100% or a physical control (slider, knob, etc.) or virtual control (indicator on an application running on a phone or tablet).

Although wearable devices have been described herein primarily with respect to providing therapeutic UV exposure at wavelengths associated with Vitamin-D synthesis in humans, the wearable device may also be configured to provide therapeutic exposures: (1) at other wavelengths specifically targeting different conditions or biomarkers, for example IR exposure for the production of nitric oxide, and (2) for other therapeutic effects, for example UV exposure for the treatment of psoriasis. More detailed discussion of the benefits of exposures at various wavelengths is provided as follows. It is envisaged that the embodiments of the wearable devices disclosed herein may be configured to gain the benefit of irradiations of skin at one or more of these wavelengths.

UVB radiation (280 nm-315 nm) is absorbed by 7-dehydrocholesterol produced in the living cells of the skin resulting in the production of previtamin D3. Furthermore, the UVB radiation is absorbed by the DNA in the skin cells resulting in stimulation of the pro-opiomelanocortin (POMC) gene which results in the production of melanocyte-stimulating hormone (MSH). This hormone is responsible for stimulating melanocytes to produce a natural sunscreen, melanin, to protect the skin from damaging effects from excessive sun exposure. The POMC gene also produces adrenocorticotropic hormone (ACTH) which in turn can stimulate the adrenal glands to produce cortisol. This gene also produces beta endorphin, the endogenous opioid peptide that is responsible for the runners high and feeling of well-being. UVB radiation has been effectively used for the treatment of psoriasis.

UVA radiation penetrates deeply into the skin and results in the release of NO which causes smooth muscle relaxation in the blood vessels causing vasodilation and lower blood pressure. It improves micro-circulation of the skin thereby enhancing wound healing especially in patients with peripheral vascular disease due to diabetes. It also effects neurotransmission in a variety of organs including the gastrointestinal tract causing gastrointestinal smooth muscle relaxation and in the brain is involved in learning and memory. UVA radiation also causes immune suppression. This decreases inflammatory skin conditions, suppresses some autoimmune diseases as well as allergic asthma. However, this suppression can also decrease resistance to some skin infectious diseases and decrease immune response to some vaccines, and by increasing the generation of free radical oxygen in the dermis UVA radiation causes cross linking of the elastic structure leading to skin damage and wrinkles.

It is also known that human cells have internal biologic clocks controlled by several genes including the period and clock genes. UVB radiation affects their expression in the skin and visible radiation penetrating deeply into our bodies may affect their activity in our heart, lungs, intestines and other organs.

The benefits of red light therapy on acne have also been demonstrated in human subjects. (Lee S Y, You C E, and Park M Y. Blue and red light combination LED phototherapy for acne vulgaris in patients with skin phototype IV. Lasers Surg Med. 2007 February; 39(2):180-188, Goldberg D J and Russell B A. Combination blue (415 nm) and red (633 nm) LED phototherapy in the treatment of mild to severe acne vulgaris. J Cosmet Laser Ther. 2006 June; 8(2):71-75.). Lee et al., found that blue and red light combination LED phototherapy is a safe and non-painful method for improvement of non-inflammatory and inflammatory acne lesions in subjects with mild to moderately severe facial acne. Goldberg et al. found a mean reduction in lesion count of 81% at a 12-week follow-up when subjects underwent eight sessions of LED phototherapy (two per week 3 days apart) alternating between 415 nm blue light and 633 nm red light from a light-emitting diode (LED)-based therapy system,

Several studies have demonstrated benefits of red light or near infrared light therapy for several different dermatologic applications such as skin rejuvenation (i.e. reduction of fine lines, wrinkles and skin roughness, collagen enhancement, tissue tightness). (Wunsch A and Matuschka K. A controlled trial to determine the efficacy of red and near-infrared light treatment in patient satisfaction, reduction of fine lines, wrinkles, skin roughness, and intradermal collagen density increase. Photomed Laser Surg. 2014 February; 32(2):93-100. Lee S Y, Park K H, Choi J W, Kwon J K, Lee D R, Shin M S, Lee J S, You C E, and Park M Y. A prospective, randomized, placebo-controlled, double-blinded, and split-face clinical study on LED phototherapy for skin rejuvenation: clinical, profilometric, histologic, ultrastructural, and biochemical evaluations and comparison of three different treatment settings. J Photochem Photobiol B. 2007 July; 88(1):51-67. Goldberg D J, Amin S, Russell B A, Phelps R, Kellett N, and Reilly L A. Combined 633-nm and 830-nm led treatment of photoaging skin. J Drugs Dermatol. 2006 September; 5(8):748-753.). Wunsch et al. studied 136 volunteers who were exposed to 611-650 nm (red light therapy; RLT) or 570-850 nm (polychromatic light; energized light technology; ELT) light compared to a control group who received no therapy in a prospective randomized controlled fashion. The evaluators who were blinded assessed clinical photography, ultrasonographic collagen density measurements, computerized digital profilometry and patient satisfaction. They found that subjects experienced significantly improved skin complexion and skin feeling. Collagen ultrasonography scans demonstrated that collagen density increased from baseline to the end of the 30 treatment trial. The ultrasonography data supported the clinical photography which revealed visible improvement changes in wrinkles and skin roughness. Russell et al., found that a combination red and near infrared LED therapy (633 nm and 830 nm wavelengths) resulted in significant improvements in facial wrinkles in human subjects after 9 and 12 weeks of therapy. (Russell B A, Kellett N, and Reilly L R. A study to determine the efficacy of combination LED light therapy (633 nm and 830 nm) in facial skin rejuvenation. J Cosmet Laser Ther. 2005 December; 7(3-4):196-200.) At similar wavelengths, Lee et al., observed beneficial skin rejuvenation effects and wrinkle reduction with LED therapy. Similar to the aforementioned studies, Goldberg et al., found that 633 and 830 nm LED therapy significantly improved the appearance of wrinkles after profilometric analysis. Furthermore, electron microscopic analysis showed thicker collagen fibers post-LED therapy, and subjects reported improvements in skin softness, smoothness and firmness.

In 22 subjects with facial rhytides (wrinkles) who received 8 light treatments over a course of 4 weeks, Sadick et al. found that the combination of red and near infrared LED therapy delivered from a small portable handheld unit resulted in improvements in fine lines and wrinkles at 8 weeks post-treatment as reported by the participants, (Sadick N S. A study to determine the efficacy of a novel handheld light-emitting diode device in the treatment of photoaged skin. J Cosmet Dermatol. 2008 December; 7(4):263-267.) Sadick et al, concluded that LED therapy may be a safe and effective method of photo rejuvenation.

In a study by Baez et al., 91% of subjects reported improved skin tone and 82% reported enhanced smoothness of the skin following 12 weeks of LED therapy at wavelengths of 633 and 830 nm. (Baez F and Reilly L R. The use of light-emitting diode therapy in the treatment of photoaged skin. J Cosmet Dermatol. 2007 September; 6(3):189-194.)

The results discussed above demonstrate beneficial effects of light therapy on wound healing, skin rejuvenation, reduced wrinkling, acne and reduced scarring. LED therapy has also been demonstrated to have an effect on the skin's immune system. (Takezaki S, Omi T, Sato S, and Kawana S. Ultrastructural observations of human skin following irradiation with visible red light-emitting diodes (leds): a preliminary in vivo report. Laser Ther. 2005; 14(4): 153-160. Takezaki S, Omi T, Sato S, and Kawana S. Light-emitting diode phototherapy at 630+/−3 nm increases local levels of skin-homing T-cells in human subjects. J Nippon Med Sch. 2006 April; 73(2):75-81.). Takezaki et al, exposed the lateral aspect of the leg of 6 adult male volunteers (ages 35-48 years) once a week for 8 weeks with visible red (630+−3 nm) and then performed a skin biopsy. They evaluated by quantitative polymerase chain reaction and by transmission electron microscopy T cell type and fibroplastic changes. They observed mild fibroplastic changes in fibroblasts with no acute inflammatory changes throughout the treatment session. Qualitative polymerase chain reaction techniques showed the presence of both Th-1 and Th-2 T cells and increased numbers of both types of skin-homing T cells. Thus, the researchers demonstrated that visible red LED irradiation activates the immune system in normal skin and thus may explain the observed effect that red light therapy has on acne, wound healing and atopic dermatitis.

Several clinical studies have demonstrated the effects of red light or near infrared light therapy on the healing of diabetic ulcers (Minatel D G, Frade M A, Franca S C, and Enwemeka C S. Phototherapy promotes healing of chronic diabetic leg ulcers that failed to respond to other therapies, Lasers Surg Med. 2009 August; 41(6):433-441, Minatel D G, Enwemeka C S, Franca S C, and Frade M A. Phototherapy (LEDs 660/890 nm) in the treatment of leg ulcers in diabetic patients: case study. An Bras Dermatol. 2009 July; 84(3):279-283.) or venous leg ulcers (Caetano K S, Frade M A, Minatel D G, Santana L A, and Enwemeka C S. Phototherapy improves healing of chronic venous ulcers. Photomed Laser Surg. 2009 February; 27(1):111-118. Gupta A K, Filonenko N, Salansky N, and Sauder D N. The use of low energy photon therapy (LEPT) in venous leg ulcers: a double-blind, placebo-controlled study. Dermatol. Surg. 1998 December; 24(12):1383-1386.). In the two studies by Minatel et al., combined 660 and 890 nm light promoted rapid granulation and healing of diabetic ulcers that had failed to respond to other forms of treatment. Caetano et al randomized 20 patients with chronic ulcers, Phototherapy with combined 660 and 890 nm light promoted significantly up to 40% faster healing of chronic venous ulcers at 30 and 90 days compared to a placebo treated control group. Similarly, in a double-blinded study, Gupta et al. found that low energy photon therapy (LEPT) (660 nm and 880 nm) three times per week for 10 weeks caused faster healing of the ulcer area in subjects with venous leg ulcers compared to the control group.

The association between red light therapy and wound healing also has been investigated. Angiogenesis is a process characterized by the formation of new blood vessels from existing ones and involves the migration, differentiation and growth of the endothelial cells, forming the wall of blood vessels. The process is especially necessary during wound healing. The effects of light irradiation at 632.8 nm in the healing process of a long-lasting radiotherapy-induced ulcer in a patient were evaluated. After 4 weeks and a total of 7 irradiation sessions, the ulcer was healed. The healing process was accompanied by a significantly increased number of dermal vessels in re-epithelialized skin compared to pretreatment conditions (Schindl A, Schindl M, Schindl L, Jureeka W, Honigsmann H and Breier F. Increased dermal angiogenesis after low-intensity laser therapy for a chronic radiation ulcer determined by a video measuring system. J Am. Academy Dermatol. 1999; 40(3): 481-484.).

According to some embodiments, a wearable device for therapeutic irradiation of skin may comprise; a light source; a light spreading sheet optically coupled to the light source, the light spreading sheet having a first surface and a second surface; a controller electrically coupled to the light source, the controller being configured for controlling the intensity of light emitted from the light source and the duration of emission of light from the light source during a therapeutic session; a proximity sensor for detecting proximity of the light spreading sheet to skin, the proximity sensor being attached to at least one of the first surface and the second surface of the light spreading sheet, the proximity sensor being electrically coupled to the controller; and a power source electrically coupled to the light source and the controller; wherein the controller is further configured to turn on, and keep turned on for the duration of the therapeutic session, the light source when the proximity sensor detects proximity of the light spreading sheet to the skin. Furthermore, the wearable device may further comprise a pigmentation sensor for measuring skin pigmentation, the pigmentation sensor being attached to the first surface of the light spreading sheet, the pigmentation sensor being electrically coupled to the controller, and wherein the controller is further configured to adjust the intensity of light and duration of light emission in response to input from the pigmentation sensor; in embodiments the pigmentation sensor may be configured for determining at least three pigmentation levels; furthermore, the wearable device may further comprise a second visible light source attached to the second surface of the light spreading sheet, the second visible light source being electrically coupled to the controller, wherein the controller activates the second visible light source when the proximity detector fails to detect proximity of the light spreading sheet to the skin; and in embodiments wherein the second visible light source may be a second plurality of LEDs; and in embodiments wherein the proximity detector may be a multiplicity of proximity detectors and the controller is configured to activate one of the second plurality of LEDs in a position on the light spreading sheet corresponding to the position of one of the multiplicity of proximity detectors in response to the one of the second plurality of proximity detectors failing to detect proximity to the skin. Furthermore, in embodiments wherein the light spreading sheet may be flexible for roughly conforming to skin on different parts of a human body. Furthermore, in embodiments wherein the light spreading sheet may comprise a plurality of panels, the plurality of panels being coupled by flexible joints for allowing the plurality of panels to roughly conform to skin on different parts of a human body. Furthermore, in embodiments wherein the light source may comprise at least one LED. Furthermore, in embodiments wherein the light source may comprise at least one laser. Furthermore, in embodiments wherein the light source may emit light in the wavelength range from 290 nm to 320 nm. Furthermore, in embodiments wherein the light source may emit infrared light. Furthermore, in embodiments wherein the power source may be a rechargeable battery. Furthermore, in embodiments wherein the light spreading sheet may be a single optical waveguide. Furthermore, in embodiments wherein the light spreading sheet may comprise optical fibers. Furthermore, in embodiments wherein the light spreading sheet may comprise a plurality of printed waveguides. Furthermore, in embodiments wherein the light source may comprise a multiplicity of light sources emitting light at a corresponding multiplicity of different wavelengths. Furthermore, in embodiments wherein the controller may have independent control of each of the multiplicity of light sources. Furthermore, in embodiments wherein the light source may comprise a first light source emitting light at first wavelengths and a second light source emitting light at second wavelengths, wherein the first wavelengths and the second wavelengths are different; in embodiments wherein the controller may have independent control of the first light source and the second light source; in embodiments wherein the first light source may comprise a multiplicity of LEDs. Furthermore, in embodiments wherein the light source may be a multiplicity of light sources and the controller is configured to independently control each of the multiplicity of light sources. Furthermore, in embodiments wherein the light source may be optically coupled to an edge of the light spreading sheet. Furthermore, the wearable device may further comprise a first visible light source attached to the second surface of the light spreading sheet, the first visible light source being electrically coupled to the controller, wherein the controller activates the first visible light source when the light source is emitting light; in embodiments wherein the first visible light source may be a first plurality of LEDs. Furthermore, the wearable device may further comprise a filter for attenuating the emission of undesirable wavelengths from the first surface of the light spreading sheet; in embodiments wherein the light source may emit light in the UV and the filter attenuates light with wavelength below 290 nm. Furthermore, the wearable device may further comprise a non-volatile memory coupled to the controller. Furthermore, the wearable device may further comprise a feedback sensor for detecting the intensity of light at a therapeutic wavelength, the feedback sensor being electrically coupled to the controller, the feedback sensor being configured within the wearable device for the detecting; in embodiments wherein the controller may adjust light intensity of the light source in response to input from the feedback sensor; in embodiments wherein the feedback sensor may comprise two or more sensors. Furthermore, in embodiments wherein the light source emits UVA light. Furthermore, in embodiments wherein the light source emits red light. Furthermore, in embodiments wherein the light source emits near IR light. Furthermore, in embodiments wherein the light source emits blue light. Furthermore, in embodiments wherein the light source emits UVB light. Furthermore, in embodiments wherein the light source emits light in the wavelength range from 321 nm to 400 nm.

According to some embodiments, a wearable device for therapeutic irradiation of skin may comprise: a substrate, the substrate having a first surface and a second surface; a light source comprising an array of light emitting diodes (LEDs) attached to the first surface of the substrate; a controller electrically coupled to the light source, the controller being configured for controlling the intensity of light emitted from the light source and the duration of emission of light from the light source during a therapeutic session; a proximity sensor for detecting proximity of the substrate to skin, the proximity sensor being attached to at least one of the first surface and the second surface of the substrate, the proximity sensor being electrically coupled to the controller; and a power source electrically coupled to the light source and the controller; wherein the controller is further configured to turn on, and keep turned on for the duration of the therapeutic session, the light source when the proximity sensor detects proximity of the light spreading sheet to the skin. Furthermore, the wearable device may further comprise a pigmentation sensor for measuring skin pigmentation, the pigmentation sensor being attached to one of the first surface and the second surface of the substrate, the pigmentation sensor being electrically coupled to the controller, and wherein the controller is further configured to adjust the intensity of light and duration of light emission in response to input from the pigmentation sensor. Furthermore, in embodiments wherein the substrate may be flexible for roughly conforming to skin on different parts of a human body. Furthermore, in embodiments wherein the substrate may comprise a plurality of panels, the plurality of panels being coupled by flexible joints for allowing the plurality of panels to roughly conform to skin on different parts of a human body. Furthermore, in embodiments wherein the light source emits light in the wavelength range from 290 nm to 320 nm. Furthermore, in embodiments wherein the light source emits infrared light. Furthermore, in embodiments wherein the power source may be a rechargeable battery. Furthermore, in embodiments wherein the light source may comprise a multiplicity of light sources emitting light at a corresponding multiplicity of different wavelengths; in embodiments wherein the controller may have independent control of each of the multiplicity of light sources; in embodiments wherein at least two of the arrays of LEDs corresponding to the multiplicity of light sources may be offset spatially from each other; in embodiments wherein at least two of the arrays of LEDs corresponding to the multiplicity of light sources have different aerial densities. Furthermore, in embodiments wherein the light source comprises a first light source emitting light at first wavelengths and a second light source emitting light at second wavelengths, wherein the first wavelengths and the second wavelengths are different; in embodiments wherein arrays of LEDs corresponding to the first light source and the second light source are offset spatially from each other; in embodiments wherein the controller has independent control of the first light source and the second light source. Furthermore, in embodiments wherein the controller may be configured to independently control each of the LEDs in the array of LEDs. Furthermore, the wearable device may further comprise a first visible light source attached to the second surface of the substrate, the first visible light source being electrically coupled to the controller, wherein the controller activates the first visible light source when the light source is emitting light; in embodiments wherein the first visible light source may be a first plurality of LEDs. Furthermore, the wearable device may further comprise a second visible light source attached to the second surface of the substrate, the second visible light source being electrically coupled to the controller, wherein the controller activates the second visible light source when the proximity detector fails to detect proximity of the substrate to the skin; in embodiments wherein the second visible light source is a second plurality of LEDs; in embodiments wherein the proximity detector is a multiplicity of proximity detectors and the controller is configured to activate one of the second plurality of LEDs in a position on the substrate corresponding to the position of one of the multiplicity of proximity detectors in response to the one of the second plurality of proximity detectors failing to detect proximity to the skin. Furthermore, the wearable device may further comprise a non-volatile memory coupled to the controller. Furthermore, the wearable device may further comprise a feedback sensor for detecting the intensity of light at a therapeutic wavelength, the feedback sensor being electrically coupled to the controller, the feedback sensor being configured within the wearable device for the detecting; in embodiments wherein the controller adjusts light intensity of the light source in response to input from the feedback sensor; in embodiments wherein the feedback sensor comprises two or more sensors. Furthermore, in embodiments wherein the array of LEDs may be an array of printed LEDs. Furthermore, in embodiments wherein the substrate may comprise a light spreading sheet optically coupled to the light source; in embodiments, the wearable device may further comprise a filter for attenuating the emission of undesirable wavelengths from the first surface of the substrate; in embodiments the light source emits light in the UV and the filter attenuates light with wavelength below 290 nm. Furthermore, in embodiments wherein the light source emits UVA light. Furthermore, in embodiments wherein the light source emits red light. Furthermore, in embodiments wherein the light source emits near IR light. Furthermore, in embodiments wherein the light source emits blue light. Furthermore, in embodiments wherein the light source emits UVB light. Furthermore, in embodiments wherein the light source emits light in the wavelength range from 321 nm to 400 nm.

According to some embodiments, a method of irradiating a patient's skin with a wearable device may comprise: providing a wearable device, the wearable device comprising a substrate, the substrate having a first surface and a second surface, a light source attached to the substrate, a controller electrically coupled to the light source, the controller being configured for controlling the intensity of light emitted from the light source and the duration of emission of light from the light source during a therapeutic session, a proximity sensor for detecting proximity of the substrate to the patient's skin, the proximity sensor being attached to at least one of the first surface and the second surface of the substrate, the proximity sensor being electrically coupled to the controller, and a power source electrically coupled to the light source and the controller; wherein the controller is further configured to only turn on, and keep turned on, the light source when the proximity sensor detects proximity of the substrate to the patient's skin; placing the wearable device in proximity to the patient's skin; detecting proximity of the light spreading sheet to the patient's skin by the controller; and on detecting proximity, turning on the light source by the controller for a prescribed time. Furthermore, the method may further comprise: after the placing and before the turning on, determining the pigmentation of the patient's skin using a pigmentation sensor in communication with the controller; and calculating by the controller of the prescribed time; wherein the wearable device comprises the pigmentation sensor. Furthermore, the method may further comprise: adjusting the light intensity of the light source by the controller in response to input from a feedback sensor; wherein the wearable device further comprises at least one feedback sensor for detecting the intensity of light at a therapeutic wavelength, the at least one feedback sensor being electrically coupled to the controller, the at least one feedback sensor being configured within the wearable device for the detecting. Furthermore, the method may further comprise: after irradiation of the patient's skin for the prescribed time, disabling the light source for a prescribed time between subsequent prescribed irradiations; wherein the wearable device further comprises a real time clock, the real time clock being electrically coupled to the controller. Furthermore, the method may further comprise, after irradiation of the patient's skin for the prescribed time, removing the wearable device from the patient; in embodiments the method may further comprise, after the removing, repeating the placing, the turning on for the prescribed time, and the removing; in embodiments the method may further comprise, after irradiation of the patient's skin for the prescribed time, disabling the light source for a second prescribed time before the repeating the turning on for the prescribed time; in embodiments wherein the repeating the placing may comprise placing the wearable device on a different area of the patient's skin, non-overlapping with the previous area of the patient's skin; in embodiments the method may further comprise, before the repeating the placing, providing, by the controller, to the patient instructions for placement of the wearable device over the different area of the patient's skin. Furthermore, in embodiments wherein the substrate may comprise a light spreading sheet and the light source is optically coupled to the light spreading sheet. Furthermore, in embodiments wherein the light source emits UVA light. Furthermore, in embodiments wherein the light source emits red light. Furthermore, in embodiments wherein the light source emits near IR light. Furthermore, in embodiments wherein the light source emits blue light. Furthermore, in embodiments wherein the light source emits UVB light. Furthermore, in embodiments wherein the light source emits light in the wavelength range from 321 nm to 400 nm.

Although embodiments of the present disclosure have been particularly described with reference to certain embodiments thereof, it should be readily apparent to those of ordinary skill in the art that changes and modifications in the form and details may be made without departing from the spirit and scope of the disclosure.

1. A wearable device for therapeutic irradiation of skin, comprising:
a light source;
a light spreading sheet optically coupled to said light source, said light spreading sheet having a first surface and a second surface;
a controller electrically coupled to said light source, said controller being configured for controlling the intensity of light emitted from said light source and duration of emission of light from said light source during a therapeutic session;
a proximity sensor for detecting proximity of said light spreading sheet to skin, said proximity sensor being attached to at least one of said first surface and said second surface of said light spreading sheet, said proximity sensor being electrically coupled to said controller; and
a power source electrically coupled to said light source and said controller;
wherein said controller is further configured to turn on, and keep turned on for said duration of said therapeutic session, said light source when said proximity sensor detects proximity of said light spreading sheet to said skin.

2. The wearable device of claim 1, further comprising a pigmentation sensor for measuring skin pigmentation, said pigmentation sensor being attached to said first surface of said light spreading sheet, said pigmentation sensor being electrically coupled to said controller, and wherein said controller is further configured to adjust the intensity of light and duration of light emission in response to input from said pigmentation sensor.

3. The wearable device of claim 1, wherein said light spreading sheet is flexible for roughly conforming to skin on different parts of a human body.

4. The wearable device of claim 1, wherein said light spreading sheet comprises a plurality of panels, said plurality of panels being coupled by flexible joints for allowing said plurality of panels to roughly conform to skin on different parts of a human body.

5. The wearable device of claim 1, wherein said light source comprises at least one LED.

6. The wearable device of claim 1, wherein said light source comprises at least one laser.

7. The wearable device of claim 1, wherein said light source emits light in the wavelength range from 290 nm to 320 nm.

8. The wearable device of claim 1, wherein said light source emits infrared light.

9. The wearable device of claim 2, wherein said pigmentation sensor is configured for determining at least three pigmentation levels.

10. The wearable device of claim 1, wherein said power source is a rechargeable battery.

11. The wearable device of claim 1, wherein said light spreading sheet is a single optical waveguide.

12. The wearable device of claim 1, wherein said light spreading sheet comprises optical fibers.

13. The wearable device of claim 1, wherein said light spreading sheet comprises a plurality of printed waveguides.

14. The wearable device of claim 1, wherein said light source comprises a multiplicity of light sources emitting light at a corresponding multiplicity of different wavelengths.

15. The wearable device of claim 14, wherein said multiplicity of light sources comprise at least a light source which emits light in the wavelength range from 290 nm to 320 nm.

16. The wearable device of claim 14, wherein said controller has independent control of each of said multiplicity of light sources.

17. The wearable device of claim 1, wherein said light source comprises a first light source emitting light at first wavelengths and a second light source emitting light at second wavelengths, wherein said first wavelengths and said second wavelengths are different.

18. The wearable device of claim 17, wherein said controller has independent control of said first light source and said second light source.

19. The wearable device of claim 17, wherein said first light source comprises a multiplicity of LEDs.

20. The wearable device of claim 1, wherein said light source is a multiplicity of light sources and said controller is configured to independently control each of said multiplicity of light sources.

21. The wearable device of claim 1, wherein said light source is optically coupled to an edge of said light spreading sheet.

22. The wearable device of claim 1, further comprising a first visible light source attached to said second surface of said light spreading sheet, said first visible light source being electrically coupled to said controller, wherein said controller activates said first visible light source when said light source is emitting light.

23. The wearable device of claim 22, wherein said first visible light source is a first plurality of LEDs.

24. The wearable device of claim 2, further comprising a second visible light source attached to said second surface of said light spreading sheet, said second visible light source being electrically coupled to said controller, wherein said controller activates said second visible light source when said proximity detector fails to detect proximity of said light spreading sheet to said skin.

25. The wearable device of claim 24, wherein said second visible light source is a second plurality of LEDs.

26. The wearable device of claim 25, wherein said proximity detector is a multiplicity of proximity detectors and said controller is configured to activate one of said second plurality of LEDs in a position on said light spreading sheet corresponding to the position of one of said multiplicity of proximity detectors in response to said one of said second plurality of proximity detectors failing to detect proximity to said skin.

27. The wearable device of claim 1, further comprising a filter for attenuating the emission of undesirable wavelengths from the first surface of said light spreading sheet.

28. The wearable device of claim 27, wherein said light source emits light in the UV and said filter attenuates light with wavelength below 290 nm.

29. The wearable device of claim 1, further comprising non-volatile memory coupled to said controller.

30. The wearable device of claim 1, further comprising a feedback sensor for detecting the intensity of light at a therapeutic wavelength, said feedback sensor being electrically coupled to said controller, said feedback sensor being configured within said wearable device for said detecting.

31. The wearable device of claim 30, wherein said controller adjusts light intensity of said light source in response to input from said feedback sensor.

32. The wearable device of claim 30, wherein said feedback sensor comprises two or more sensors.

33-60. (canceled)

61. A method of irradiating a patient's skin with a wearable device, comprising:
providing a wearable device, said wearable device comprising:
a substrate, said substrate having a first surface and a second surface;
a light source attached to said substrate, wherein said substrate comprises a light spreading sheet and said light source is optically coupled to said light spreading sheet;
a controller electrically coupled to said light source, said controller being configured for controlling the intensity of light emitted from said light source and the duration of emission of light from said light source during a therapeutic session;
a proximity sensor for detecting proximity of said substrate to said patient's skin, said proximity sensor being attached to at least one of said first surface and said second surface of said substrate, said proximity sensor being electrically coupled to said controller; and
a power source electrically coupled to said light source and said controller;
wherein said controller is further configured to only turn on, and keep turned on, said light source when said proximity sensor detects proximity of said substrate to said patient's skin;
placing said wearable device in proximity to said patient's skin;
detecting proximity of said light spreading sheet to said patient's skin by said controller; and
on detecting proximity, turning on said light source by said controller for a prescribed time.

62. The method of claim 61, further comprising:
after said placing and before said turning on, determining the pigmentation of said patient's skin using a pigmentation sensor in communication with said controller; and
calculating by said controller of the prescribed time;
wherein said wearable device comprises said pigmentation sensor.

63. The method of claim 61, further comprising:
adjusting the light intensity of said light source by said controller in response to input from a feedback sensor;
wherein said wearable device further comprises at least one feedback sensor for detecting the intensity of light at a therapeutic wavelength, said at least one feedback sensor being electrically coupled to said controller, said at least one feedback sensor being configured within said wearable device for said detecting.

64. The method of claim 61, further comprising:
after irradiation of said patient's skin for said prescribed time, disabling said light source for a prescribed time between subsequent prescribed irradiations;
wherein said wearable device further comprises a real time clock, said real time clock being electrically coupled to said controller.

65. The method of claim 61, further comprising, after irradiation of said patient's skin for said prescribed time, removing said wearable device from said patient.

66. The method of claim 65, further comprising, after said removing, repeating said placing, said turning on for said prescribed time, and said removing.

67. The method of claim 66, further comprising, after irradiation of said patient's skin for said prescribed time, disabling said light source for a second prescribed time before said repeating said turning on for said prescribed time.

68. The method of claim 66, wherein said repeating said placing comprises placing said wearable device on a different area of said patient's skin, non-overlapping with the previous area of said patient's skin.

69. The method of claim 68, further comprising, before said repeating said placing, providing, by said controller, to said patient instructions for placement of said wearable device over said different area of said patient's skin.

70. (canceled)

71. The wearable device of claim 1, wherein said light source emits UVA light.

72. The wearable device of claim 1, wherein said light source emits red light.

73. The wearable device of claim 1, wherein said light source emits near IR light.

74. The wearable device of claim 1, wherein said light source emits blue light.

75. The wearable device of claim 1, wherein said light source emits UVB light.

76. The wearable device of claim 1, wherein said light source emits light in the wavelength range from 321 nm to 400 nm.
